(19) United States
(12) Patent Application Publication
(10) Pub. No.: US 2005/0256552 A1
(43) Pub. Date: Nov. 17, 2005

(54) Toenail fungus eradicator

(76) Inventor: Robert Lee White, Kannapolis, NC (US)

Correspondence Address: Robert Lee White, 400 Dakota Street, Kannapolis, NC, 28083, US

(21) Appl. No.: 10/657,571

(22) Filed: May 17, 2004

(51) Int. Cl. A61N 5/06

(52) U.S. Cl. 607/88

(57) ABSTRACT

This is a device designed to illuminate a toenail or fingernail fungal infection for extended periods of time for the purpose of eradicating that infection. The device is battery powered and can be comfortably worn while sleeping (as would be probable in the case of toenail infections), or can be worn continuously (as would be probable in the case of fingernail infections). The prolonged exposure to light will lead to the death of the fungus. This is non-invasive, and totally safe for the wearer. The other effective method of treatment available consists of the ingestion of potentially toxic medication for extended periods of time.

The invention is a very simple device consisting of a light emitting diode, a plastic housing, a round battery, (such as a hearing aid or computer motherboard battery), and the necessary wiring to complete the electrical circuit between the two, thus powering the diode which then emits light.

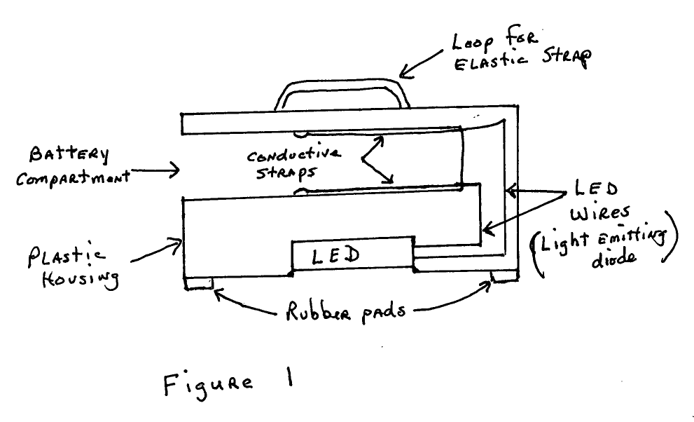

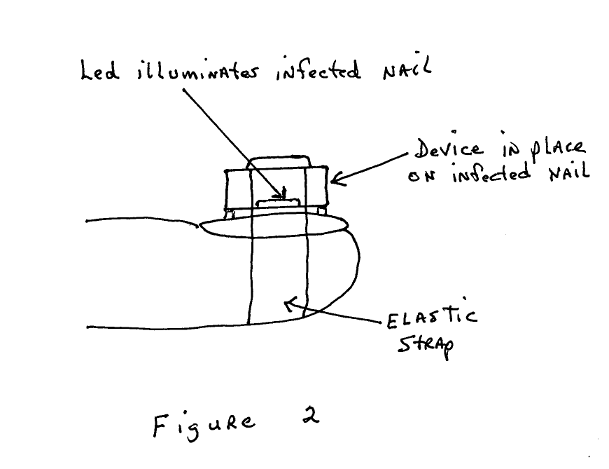

(See drawing FIG. 1).

Note: (FIG. 1 shows the device without the battery inserted. Inserting the battery and removing the battery powers the device on and off.) Also, this device can be made in various sizes to better fit the infected nail.

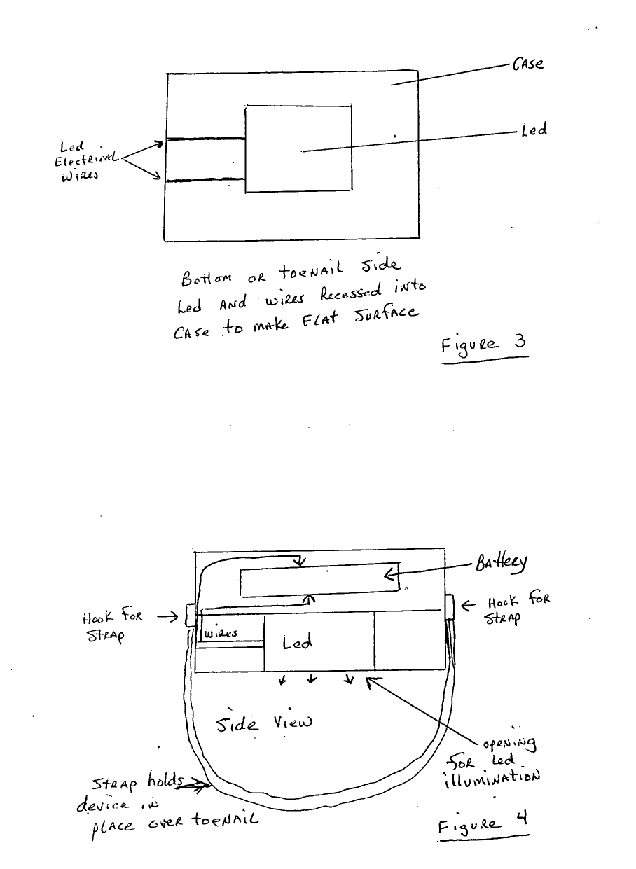

The light from the diode is directed toward the infected nail by an opening in the plastic housing. The device is attached to the infected toenail or fingernail with an elastic strap threaded though a loop built into the plastic housing. (See FIG. 2)

The purpose of this invention is the safe and effective treatment of nail bed fungal infections using light. This is a non-invasive alternative to conventional methods of treatment. Conventional methods of treatment consist of topical creams, (which do not reach the infection and are thus ineffective), and systemic medications (which do reach the infection, but have systemic toxic properties, and are expensive to use).

The method I propose is totally non-invasive and totally safe, and very inexpensive.

To the best of my knowledge, I am the first person to think of using a “wearable” battery operated device which uses light to treat fungal infections of toenails and or fingernails.

I believe that all spectrums or colors of light may be effective in the treatment of fungal infections, but the use of a ultra-violet light emitting diode may be required for severe infections. It is well documented that fungi do not thrive in direct light and this method of treatment will ensure that they are exposed to direct light for several hours at a time or continuously, as tolerated by the wearer of the device. This device can be worn while sleeping, (as would probably be the case for toenail infections as it would be difficult to wear inside your shoes) or continuously, as would probably be the case in fingernail infections. I believe that this method is superior to conventional methods of treatment in terms of safety and cost.

1. To the best of my knowledge, I am the first to think of using a “wearable” battery powered light emitting diode device to directly illuminate toenail and fingernail fungal infections for the purpose of eradicating those fungal infections. This device fits over the infected area and exposes the fungal infection to light for extended periods of time. Prolonged exposure to light is fatal to most fungi.

2. I propose using a very simple device consisting of a small battery (such as a hearing aid battery or computer motherboard battery), a light emitting diode, a plastic housing, and the necessary wiring to connect them together to complete a simple circuit making the diode emit light, and a method to attach them to the infected nail. The attachment may be an elastic strap, Velcro strap, or any other method which will hold the device in place. (see drawing FIG. 2)

3. I would like this patent to cover all spectrums and colors of light, including ultraviolet, as I am unsure which spectrum/color is most effective.

4. This is a vast improvement over the current practice of using systemic medications that are potentially harmful to the liver and other organs, and are also extremely expensive.

5. This improved technique is cheaper, and safer, as it is non-invasive and has no predicted systemic effects.
